(19) United States
(12) Patent Application Publication
(10) Pub. No.: US 2024/0219362 A1
(43) Pub. Date: Jul. 4, 2024

(54) METHOD FOR DETECTING POPULATION DENSITY OF CRYPTOLESTES FERRUGINEUS BASED ON CO2 RELEASE RATE

(71) Applicant: Nanjing University of Finance & Economics, Nanjing (CN)

(72) Inventors: Pei'an Tang, Nanjing (CN); Xueyou Wu, Nanjing (CN); Kangxu Wang, Nanjing (CN); Shengyuan Sun, Nanjing (CN); Yan Chen, Nanjing (CN)

(21) Appl. No.: 18/170,321

(22) Filed: Feb. 16, 2023

(30) Foreign Application Priority Data
Dec. 15, 2022 (CN) 202211626195.1

(51) Int. Cl. A01M 31/00; G06Q 10/04

(52) U.S. Cl. CPC A01M 31/002; G06Q 10/04; A01M 2200/012

(57) ABSTRACT

The present disclosure provides a method for detecting a population density of Cryptolestes ferrugineus based on a CO₂ release rate, and belongs to the technical field of pest detection in stored grains. In the present disclosure, the method includes: constructing a prediction model of a population density of Cryptolestes ferrugineus based on a relationship between different stored grain temperatures, stored grain water contents, and CO₂ release rates in the environment and the population density of the Cryptolestes ferrugineus; measuring the stored grain temperature and the CO₂ release rate in the environment, substituting measured values into the prediction model of the population density of the Cryptolestes ferrugineus for calculation to obtain the population density of the Cryptolestes ferrugineus in a grain storage environment; and determining a pest-carrying grain grade. The method can eliminate an interference of dead pests and death-feigning pests, and can also detect borer pests.

### CROSS REFERENCE TO RELATED APPLICATION

This patent application claims the benefit and priority of Chinese Patent Application No. 202211626195.1, filed with the China National Intellectual Property Administration on Dec. 15, 2022, the disclosure of which is incorporated by reference herein in its entirety as part of the present application.

### TECHNICAL FIELD

The present disclosure belongs to the technical field of pest detection in stored grains, and in particular relates to a method for detecting a population density of Cryptolestes ferrugineus based on a CO₂ release rate.

### BACKGROUND

Stored grain pests are one of the important biological factors that cause the loss of grain quantity and quality. Traditionally, the stored grain pests are mainly monitored by manual sampling method and trap method, which are time-consuming, laborious, and narrow in scope of application; moreover, when pest-carrying grains are detected, huge losses have generally been caused to the stored grains. Compared with these traditional monitoring methods for stored grain pests, intelligent and early-warning monitoring technology can help to grasp the occurrence of stored grain pests in a more timely and accurate manner, thereby helping grain depots prevent the occurrence of stored grain pests to protect the safety of stored grains. Accordingly, it has become an inevitable trend by constructing an intelligent grain depot.

At present, several existing intelligent monitoring technologies have their own deficiencies. For example, the image monitoring method can only automatically identify active pests outside the grains, but cannot differentiate borer pests, death-feigning pests, and larvae from each other; the capacitive sensor method has a low efficiency; the infrared photoelectric technology is sensitive to the humidity of samples, and is not easy to identify pests with similar body shapes; and the acoustic detection method needs to remove the influence of environmental noise. Therefore, there is an urgent need to develop a set of intelligent detection technology that is widely used, easy to operate, and highly feasible, and can be used for early detection of stored grain pests.

### SUMMARY

In view of this, an objective of the present disclosure is to provide a method for detecting a population density of Cryptolestes ferrugineus based on a CO₂ release rate. In the present disclosure, a theoretical basis is provided for monitoring the pest-carrying grain situation using CO₂ by establishing a monitoring model for a population density of the Cryptolestes ferrugineus, and a new method is established for the early monitoring of stored grain pests.

The present disclosure provides a method for detecting a population density of Cryptolestes ferrugineus based on a CO₂ release rate, including the following steps:
constructing a prediction model of a population density of Cryptolestes ferrugineus based on a relationship between different stored grain temperatures, stored grain water contents, and CO₂ release rates in the environment and the population density of the Cryptolestes ferrugineus; and
measuring the stored grain temperature and the CO₂ release rate in the environment, substituting measured values into the prediction model of the population density of the Cryptolestes ferrugineus for calculation to obtain the population density of the Cryptolestes ferrugineus in a grain storage environment.

Preferably, when the stored grain water content is 11.5% to 12.5%, the prediction model of the population density of the Cryptolestes ferrugineus is shown in formula I:

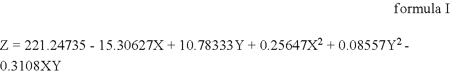

where, R²=0.94022; X is the temperature in ° C.; Y is the CO₂ release rate in ppm/h; and Z is the population density of the Cryptolestes ferrugineus in insects/kg.

Preferably, when the stored grain water content is 12.6% to 13.5%, the prediction model of the population density of the Cryptolestes ferrugineus is shown in formula II:

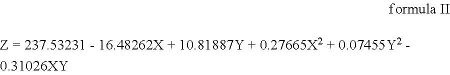

where, R²=0.93665; X is the temperature in ° C.; Y is the CO₂ release rate in ppm/h; and Z is the population density of the Cryptolestes ferrugineus in insects/kg.

Preferably, when the stored grain water content is 13.6% to 14.5%, the prediction model of the population density of the Cryptolestes ferrugineus is shown in formula III:

Z=237.71237−16.53808X+10.38359Y+0.27835X²+0.07426Y²−0.30076XY formula III
where, R²=0.93604; X is the temperature in ° C.; Y is the CO₂ release rate in ppm/h; and Z is the population density of the Cryptolestes ferrugineus in insects/kg.

Preferably, the temperature includes 25° C. to 35° C.

Preferably, in the Cryptolestes ferrugineus, adults and larvae are at a quantity ratio of 1:(4.93-8.37).

Preferably, the stored grain includes wheat.

Preferably, a pest in the stored grain is the Cryptolestes ferrugineus.

The present disclosure further provides use of a method for detecting a population density of Cryptolestes ferrugineus based on a CO₂ release rate in determination of a pest-carrying grain grade.

Preferably, the pest-carrying grain grade includes a grade of basically no pest-carrying grain, a grade of general pest-carrying grain, and a grade of serious pest-carrying grain in unprocessed wheat grains.

The present disclosure provides a method for detecting a population density of Cryptolestes ferrugineus based on a CO₂ release rate, including the following steps: constructing a prediction model of a population density of Cryptolestes ferrugineus based on a relationship between different stored grain temperatures, stored grain water contents, and CO₂ release rates in the environment and the population density of the Cryptolestes ferrugineus; and measuring the stored grain temperature and the CO₂ release rate in the environment, substituting measured values into the prediction model of the population density of the Cryptolestes ferrugineus for calculation to obtain the population density of the Cryptolestes ferrugineus in a grain storage environment. In the present disclosure, the method is to detect the quantity of the Cryptolestes ferrugineus based on a CO₂ release rate of the Cryptolestes ferrugineus; by clarifying factors that affect the CO₂ release rate, a detection model of the population density of the Cryptolestes ferrugineus is constructed. The method can eliminate an interference of dead pests and death-feigning pests, and can also detect borer pests. The method has efficient, accurate, and convenient detection, which is suitable for large-scale promotion and application.

### DETAILED DESCRIPTION OF THE EMBODIMENTS

The present disclosure provides a method for detecting a population density of Cryptolestes ferrugineus based on a CO₂ release rate, including the following steps:

constructing a prediction model of a population density of Cryptolestes ferrugineus based on a relationship between different stored grain temperatures, stored grain water contents, and CO₂ release rates in the environment and the population density of the Cryptolestes ferrugineus; and

measuring the stored grain temperature and the CO₂ release rate in the environment, substituting measured values into the prediction model of the population density of the Cryptolestes ferrugineus for calculation to obtain the population density of the Cryptolestes ferrugineus in a grain storage environment.

In the present disclosure, a prediction model of a population density of Cryptolestes ferrugineus is constructed based on a relationship between different stored grain temperatures, stored grain water contents, and CO₂ release rates in the environment and the population density of the Cryptolestes ferrugineus.

In the present disclosure, although existing studies have confirmed that the CO₂ content in a closed grain storage environment may accumulate and increase with the breeding of stored grain pests, there is no clear result on a specific relationship between the population density of the Cryptolestes ferrugineus and the CO₂ content. Based on this, the factors that affect the population density of the Cryptolestes ferrugineus are screened. The results show that the stored grain temperature, stored grain water content, and stored grain pest species all affect the CO₂ respiration rate in the grain storage environment. Since the method is only aimed at the grain storage granary where the Cryptolestes ferrugineus breaks out, the influence of the stored grain pest species is excluded. Meanwhile, the respiration of the stored grain itself in the granary is extremely weak, and its interference is deducted. The method covers a wheat water content of 11.5% to 14.5%. In addition, the stored grain temperature is preferably 25° C. to 35° C.

In the present disclosure, different stored grain water contents mean the different prediction models of the population density of the Cryptolestes ferrugineus:

When the stored grain water content is preferably 11.5% to 12.5%, the prediction model of the population density of the Cryptolestes ferrugineus is preferably shown in formula I:

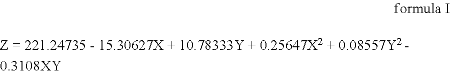

where, R²=0.94022; X is the temperature in ° C.; Y is the CO₂ release rate in ppm/h; and Z is the population density of the Cryptolestes ferrugineus in insects/kg. The stored grain water content is more preferably 12%.

When the stored grain water content is preferably 12.6% to 13.5%, the prediction model of the population density of the Cryptolestes ferrugineus is preferably shown in formula II:

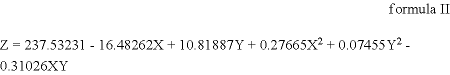

where, R²=0.93665; X is the temperature in ° C.; Y is the CO₂ release rate in ppm/h; and Z is the population density of the Cryptolestes ferrugineus in insects/kg. The stored grain water content is more preferably 13%.

When the stored grain water content is preferably 13.6% to 14.5%, the prediction model of the population density of the Cryptolestes ferrugineus is preferably shown in formula III:

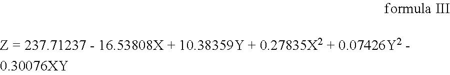

where, R²=0.93604; X is the temperature in ° C.; Y is the CO₂ release rate in ppm/h; and Z is the population density of the Cryptolestes ferrugineus in insects/kg. The stored grain water content is more preferably 14%.

In the present disclosure, the ratio of adults to larvae is also a key factor affecting the CO₂ release rate of the Cryptolestes ferrugineus. The results show that the respiration rate of adults is significantly higher than that of larvae. According to the report of Guangkai Hao et al. (Guangkai Hao, Ling Zeng, Chuanzhong Lao, and Ling Zeng. Effects of Temperature on the Growth, Development and Population Changes of the Cryptolestes ferrugineus [J]. Grain Storage, 2015, 44 (1): 1-5.), the ratio of adults and larvae of the stored grain pest Cryptolestes ferrugineus population is 1:6.62, 1:8.37, and 1:4.93 at 25° C., 30° C., and 35° C., respectively. In the Cryptolestes ferrugineus, adults and larvae are at a quantity ratio of preferably 1:(4.93-8.37).

In the present disclosure, the stored grain is preferably wheat. The prediction model of the population density of the Cryptolestes ferrugineus constructed by the present disclosure is verified. The results show that the population density of the Cryptolestes ferrugineus predicted by the method is relatively close to the actual detected number of the Cryptolestes ferrugineus, indicating that the method has high detection accuracy and easy operation, and is suitable for large-scale promotion and application.

In view of this, the present disclosure further provides use of a method for detecting a population density of Cryptolestes ferrugineus based on a CO₂ release rate in determination of a pest-carrying grain grade.

In the present disclosure, the pest-carrying grain grade includes preferably a grade of basically no pest-carrying grain, a grade of general pest-carrying grain, and a grade of serious pest-carrying grain in unprocessed wheat grains. A determination method of the pest-carrying grain grade is preferably determined according to the grade division and grade index of pest-carrying grain grade in “GB/T 29890-2013, Technical Criterion for Grain and Oil-Seeds Storage”. Specifically, the grades of wheat infested by the Cryptolestes ferrugineus are divided into: ≤5 insects/Kg is the grade of basically no pest-carrying grain, 6 insects/Kg to 30 insects/Kg (including the endpoint value) is the grade of general pest-carrying grain, and >30 insects/Kg is the grade of serious pest-carrying grain.

The method for detecting a population density of Cryptolestes ferrugineus based on a CO₂ release rate provided by the present disclosure are described in detail below with reference to the examples, but the examples cannot be understood as limiting the protection scope of the present disclosure.

### Example 1

A method for constructing a prediction model of a population density of a stored grain pest Cryptolestes ferrugineus

### 1. Test Insect Culture

The test insect, Cryptolestes ferrugineus, was bred and maintained by the Ecological Grain Storage Laboratory of Nanjing University of Finance and Economics. 200 test insects were randomly selected and placed in a 500 mL wide-mouth bottle containing 150 g of feed (including broken wheat grains and a yeast powder at a ratio of 9:1), and then incubated at (30±0.5)° C. with a relative humidity of 75% in an incubator in the dark. The adults were removed 3 d after the spawning; the eggs were kept in the feed and continued to cultivate (eggs within 3 d were regarded as a same instar), and the offspring produced were the test insects of the same instar; the test insects of corresponding states were selected for relevant tests.

2. CO₂ Release Rate of Cryptolestes ferrugineus with Different Populations Under Different Environmental Conditions

2 Kg of heat with different water contents (12%, 13%, and 14%) was separately placed in a 5 L glass vessel, and 0, 2, 5, 10, 20, and 30 adults and larvae of Cryptolestes ferrugineus were inoculated, respectively; where adults with even-numbered populations were set at a sex ratio of 1:1, while adults with odd-numbered populations were set at a sex ratio of 3:2. A three-in-one and data storage-type CO₂ detector (model B1010, purchased from Shenzhen WOST Technology Co., Ltd.) into the glass vessel, and sealed; a respiration rate of the Cryptolestes ferrugineus was monitored under the stored grain temperatures of 25° C., 30° C. and 35° C. separately; the three-in-one and data storage-type CO₂ detector was set to record data every 1 h, and the data was recorded continuously for 3 d. Table 1 and Table 2 showed results of the CO₂ release rate of Cryptolestes ferrugineus with different populations under different environmental conditions.

TABLE 1
CO₂ release rate of Cryptolestes ferrugineus adults with different
populations under different environmental conditions

| | Wheat | | | |
|---|---|---|---|---|
| Population/ | water | CO₂ release rate (ppm/h) | | |
| insects | content/% | 25° C. | 30° C. | 35° C. |
| 0 | 12 | 0.41 ± 0.055 | 3.13 ± 0.066 | 3.44 ± 0.125 |
| 2 | 12 | 1.46 ± 0.055 | 5.14 ± 0.184 | 6.29 ± 0.122 |
| 5 | 12 | 1.63 ± 0.086 | 8.10 ± 0.072 | 9.07 ± 0.108 |
| 10 | 12 | 3.02 ± 0.112 | 9.13 ± 0.102 | 12.88 ± 0.159 |
| 20 | 12 | 4.73 ± 0.122 | 11.46 ± 0.098 | 15.72 ± 0.410 |
| 30 | 12 | 6.65 ± 0.072 | 14.73 ± 0.158 | 21.90 ± 0.231 |
| 0 | 13 | 0.58 ± 0.046 | 3.47 ± 0.075 | 3.58 ± 0.049 |
| 2 | 13 | 1.71 ± 0.101 | 5.72 ± 0.026 | 6.75 ± 0.155 |
| 5 | 13 | 1.77 ± 0.027 | 8.61 ± 0.167 | 9.41 ± 0.066 |
| 10 | 13 | 3.14 ± 0.007 | 9.41 ± 0.126 | 13.05 ± 0.021 |
| 20 | 13 | 4.80 ± 0.119 | 11.74 ± 0.095 | 16.70 ± 0.005 |
| 30 | 13 | 6.82 ± 0.067 | 14.95 ± 0.047 | 22.76 ± 0.089 |
| 0 | 14 | 0.68 ± 0.013 | 3.69 ± 0.072 | 3.78 ± 0.106 |
| 2 | 14 | 1.95 ± 0.091 | 5.99 ± 0.137 | 7.19 ± 0.201 |
| 5 | 14 | 2.02 ± 0.023 | 8.97 ± 0.026 | 9.66 ± 0.140 |
| 10 | 14 | 3.34 ± 0.034 | 9.70 ± 0.212 | 13.20 ± 0.176 |
| 20 | 14 | 4.92 ± 0.122 | 11.96 ± 0.073 | 17.32 ± 0.407 |
| 30 | 14 | 7.05 ± 0.051 | 15.18 ± 0.149 | 23.44 ± 0.153 |

TABLE 2
CO₂ release rate of Cryptolestes ferrugineus larvae with different
populations under different environmental conditions

| | Wheat | | | |
|---|---|---|---|---|
| Population/ | water | CO₂ release rate (ppm/h) | | |
| insects | content/% | 25° C. | 30° C. | 35° C. |
| 0 | 12 | 0.41 ± 0.055 | 3.13 ± 0.066 | 3.44 ± 0.125 |
| 2 | 12 | 0.87 ± 0.053 | 3.46 ± 0.057 | 4.65 ± 0.109 |
| 5 | 12 | 1.41 ± 0.024 | 4.76 ± 0.023 | 5.78 ± 0.035 |
| 10 | 12 | 2.30 ± 0.044 | 6.99 ± 0.077 | 8.07 ± 0.109 |
| 20 | 12 | 3.09 ± 0.044 | 7.49 ± 0.414 | 10.04 ± 0.184 |
| 30 | 12 | 3.44 ± 0.040 | 9.30 ± 0.130 | 12.14 ± 0.754 |
| 0 | 13 | 0.58 ± 0.046 | 3.47 ± 0.075 | 3.58 ± 0.049 |
| 2 | 13 | 0.97 ± 0.006 | 3.71 ± 0.147 | 4.90 ± 0.052 |
| 5 | 13 | 1.49 ± 0.042 | 5.40 ± 0.184 | 6.29 ± 0.119 |
| 10 | 13 | 2.52 ± 0.092 | 7.28 ± 0.081 | 8.18 ± 0.121 |
| 20 | 13 | 3.32 ± 0.113 | 8.14 ± 0.154 | 10.46 ± 0.343 |
| 30 | 13 | 3.47 ± 0.015 | 9.60 ± 0.080 | 12.99 ± 0.152 |
| 0 | 14 | 0.68 ± 0.013 | 3.69 ± 0.072 | 3.78 ± 0.106 |
| 2 | 14 | 1.00 ± 0.035 | 3.99 ± 0.035 | 5.18 ± 0.054 |
| 5 | 14 | 1.78 ± 0.180 | 5.93 ± 0.112 | 6.73 ± 0.212 |
| 10 | 14 | 2.69 ± 0.071 | 7.57 ± 0.212 | 8.46 ± 0.062 |
| 20 | 14 | 3.59 ± 0.095 | 8.48 ± 0.203 | 11.48 ± 0.460 |
| 30 | 14 | 3.67 ± 0.119 | 10.34 ± 0.507 | 13.38 ± 0.137 |

The above experimental results showed that (1) the CO₂ release rate of pest-free grains with different water contents (12%, 13%, and 14%) at different storage temperatures (25° C., 30° C., and 35° C.) was much lower than that of pest-infested grains; (2) under the same grain storage environment (same grain water content and storage temperature), the stored grain pest Cryptolestes ferrugineus showed different CO₂ release rates in different stages (adults and larvae), and the CO₂ release rate of adult in respiration was larger than that of the larvae; (3) under the same grain storage environment (same grain water content and storage temperature), the CO₂ release rate in the storage environment gradually increased with an increase of the population of Cryptolestes ferrugineus infected in wheat; (4) the stored grain water content and stored grain temperature significantly affected the CO₂ release rate of the stored grain pest Cryptolestes ferrugineus, showing a positive correlation. Therefore, in the storage of raw wheat grains, the stored grain water content and the stored grain temperature each had a significant impact on the CO₂ release rate of the stored grain pest Cryptolestes ferrugineus. However, CO₂ produced by respiration of wheat itself accounted for a highly small proportion of the total CO₂ release rate in pest-infested grain storage environment, especially in the case of general pest-carrying grain and serious pest-carrying grain. In addition, the impact of the different stages, adults and larvae, of the stored grain pest Cryptolestes ferrugineus, should also be considered in the establishment of the method for detecting the population density of the Cryptolestes ferrugineus.

3. Establishment of a Prediction Model of a Population Density of the Stored Grain Pest Cryptolestes ferrugineus

Based on the above experimental results of CO₂ release rate under different environmental conditions for the different Cryptolestes ferrugineus populations, and combined with the ratios of larvae to adults in the initially stable population of Cryptolestes ferrugineus at 1:6.62, 1:8.37, and 1:4.93 under 25° C., 30° C., and 35° C., respectively (Guangkai Hao, Ling Zeng, Chuanzhong Lao, Ling Zeng. Effects of Temperature on the Growth, Development and Population Changes of the Cryptolestes ferrugineus [J]. Grain Storage, 2015, 44 (1): 1-5.), a prediction model of the population density of the Cryptolestes ferrugineus was constructed using OriginLab software based on a non-linear least squares method of the Levernberg-Marquardt algorithm (LMA).

Fitting a 12% wheat water content, the prediction model of the population density of the Cryptolestes ferrugineus was:

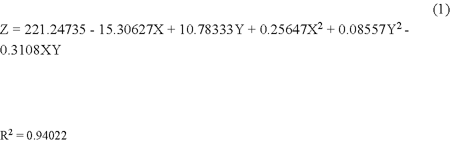

X is the temperature (° C.); Y is the CO₂ release rate (ppm/h); and Z is the population density of the Cryptolestes ferrugineus (insects/kg).

Fitting a 13% wheat water content, the prediction model of the population density of the Cryptolestes ferrugineus was:

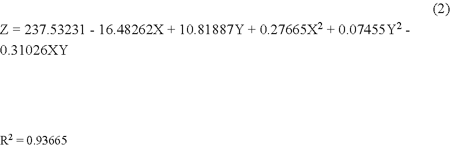

X is the temperature (° C.); Y is the CO₂ release rate (ppm/h); and Z is the population density of the Cryptolestes ferrugineus (insects/kg).

Fitting a 14% wheat water content, the prediction model of the population density of the Cryptolestes ferrugineus was:

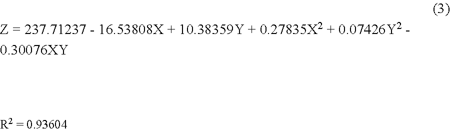

X is the temperature (° C.); Y is the CO₂ release rate (ppm/h); and Z is the population density of the Cryptolestes ferrugineus (insects/kg).

### Example 2

Verification of a Prediction Model of a Population Density of the Stored Grain Pest Cryptolestes ferrugineus

A pest-carrying grain grade is determined according to the grade division and grade index of pest-carrying grain grade in “GB/T 29890-2013, Technical Criterion for Grain and Oil-Seeds Storage”. Specifically, the grades of wheat infested by the Cryptolestes ferrugineus are divided into: ≤5 insects/Kg is the grade of basically no pest-carrying grain, 6 insects/Kg to 30 insects/Kg (including the endpoint value) is the grade of general pest-carrying grain, and >30 insects/Kg is the grade of serious pest-carrying grain.

Model validation was conducted in the Ecological Grain Storage Laboratory of Nanjing University of Finance and Economics. A three-in-one and data storage-type CO₂ detector (the same as that in Example 1) was placed in simulated grain storage tanks of different pest-carrying grain grades (5 L, containing 2 Kg of wheat, and the only pest infected with the stored grain was the Cryptolestes ferrugineus). The airtight experimental device was placed under different stored grain temperatures to monitor the respiration rate of the Cryptolestes ferrugineus, and the population density of the Cryptolestes ferrugineus was calculated. Meanwhile, according to an inspection method of stored grain pests in “GB/T 29890-2013, Technical Criterion for Grain and Oil-Seeds Storage”, the screening method, the population density of the Cryptolestes ferrugineus was manually inspected. The experimental results were shown in Table 3.

TABLE 3
Comparison between prediction and manual re-inspection of population
density of Cryptolestes ferrugineus in grain storage environment

| | | | CO₂ | Population density (insects/kg) | | | |
|---|---|---|---|---|---|---|---|
| | Wheat | | release | | Manual | | |
| | water | Temperature | rate | Model | inspection | | |
| SN | content (%) | (° C.) | (ppm/h) | results | results | Error % | Grade |
| 1 | 11.9 | 26.8 | 17.376 | 64 | 69 | 7.25 | Serious pest-carrying grain |
| 2 | 11.5 | 26.4 | 4.898 | 11 | 11 | 0.00 | General pest-carrying grain |
| 3 | 11.6 | 25.9 | 6.590 | 19 | 21 | 9.52 | General pest-carrying grain |
| 4 | 12.1 | 25.1 | 13.627 | 55 | 52 | 5.77 | Serious pest-carrying grain |
| 5 | 11.7 | 25.5 | 6.598 | 20 | 22 | 9.09 | General pest-carrying grain |
| 6 | 11.9 | 25.9 | 3.981 | 9 | 10 | 10.00 | General pest-carrying grain |
| 7 | 12.2 | 26.7 | 4.913 | 10 | 12 | 16.67 | General pest-carrying grain |
| 8 | 11.9 | 25.4 | 7.378 | 24 | 26 | 7.69 | General pest-carrying grain |
| 9 | 11.9 | 25.2 | 9.820 | 36 | 38 | 5.26 | Serious pest-carrying grain |
| 10 | 11.5 | 25.0 | 2.665 | 8 | 7 | 14.29 | General pest-carrying grain |
| 11 | 12.0 | 26.8 | 15.969 | 56 | 67 | 16.42 | Serious pest-carrying grain |
| 12 | 12.1 | 25.5 | 3.144 | 8 | 9 | 11.11 | General pest-carrying grain |
| 13 | 12.1 | 26.8 | 9.777 | 27 | 37 | 27.03 | Serious pest-carrying grain |
| 14 | 12.0 | 26.1 | 9.222 | 28 | 32 | 12.50 | Serious pest-carrying grain |
| 15 | 12.1 | 25.1 | 5.545 | 18 | 18 | 0.00 | General pest-carrying grain |
| 16 | 12.1 | 25.9 | 2.211 | 3 | 4 | 25.00 | Basically no pest-carrying grain |
| 17 | 11.6 | 25.3 | 1.583 | 3 | 4 | 25.00 | Basically no pest-carrying grain |
| 18 | 12.4 | 25.8 | 8.792 | 28 | 31 | 9.68 | Serious pest-carrying grain |
| 19 | 11.9 | 25.8 | 3.022 | 6 | 7 | 14.29 | General pest-carrying grain |
| 20 | 12.5 | 26.4 | 2.766 | 4 | 4 | 0.00 | Basically no pest-carrying grain |
| 21 | 12.2 | 29.2 | 5.414 | 5 | 5 | 0.00 | Basically no pest-carrying grain |
| 22 | 12.3 | 31.4 | 11.325 | 16 | 20 | 20.00 | General pest-carrying grain |
| 23 | 12.2 | 27.1 | 12.640 | 38 | 37 | 2.70 | Serious pest-carrying grain |
| 24 | 11.9 | 29.9 | 5.159 | 3 | 2 | 50.00 | Basically no pest-carrying grain |
| 25 | 12.2 | 32.6 | 20.494 | 44 | 47 | 6.38 | Serious pest-carrying grain |
| 26 | 12.2 | 31.9 | 15.489 | 28 | 30 | 6.67 | General pest-carrying grain |
| 27 | 12.4 | 30.0 | 17.196 | 43 | 42 | 2.38 | Serious pest-carrying grain |
| 28 | 12.1 | 32.4 | 13.745 | 21 | 23 | 8.70 | General pest-carrying grain |
| 29 | 12.4 | 28.1 | 19.609 | 67 | 66 | 1.52 | Serious pest-carrying grain |
| 30 | 12.0 | 31.9 | 8.541 | 8 | 9 | 11.11 | General pest-carrying grain |
| 31 | 11.8 | 31.1 | 9.621 | 12 | 12 | 0.00 | General pest-carrying grain |
| 32 | 12.0 | 28.1 | 6.880 | 12 | 11 | 9.09 | General pest-carrying grain |
| 33 | 12.1 | 28.1 | 20.263 | 70 | 72 | 2.78 | Serious pest-carrying grain |
| 34 | 12.1 | 31.4 | 6.071 | 3 | 2 | 50.00 | Basically no pest-carrying grain |
| 35 | 11.5 | 30.0 | 12.849 | 26 | 25 | 4.00 | General pest-carrying grain |
| 36 | 11.5 | 28.5 | 6.796 | 10 | 9 | 11.11 | General pest-carrying grain |
| 37 | 11.7 | 30.3 | 16.881 | 40 | 41 | 2.44 | Serious pest-carrying grain |
| 38 | 12.3 | 27.1 | 7.902 | 19 | 10 | 90.00 | General pest-carrying grain |
| 39 | 12.1 | 31.6 | 6.219 | 3 | 4 | 25.00 | Basically no pest-carrying grain |
| 40 | 11.9 | 30.2 | 21.638 | 63 | 66 | 4.55 | Serious pest-carrying grain |
| 41 | 11.7 | 35.0 | 17.035 | 23 | 22 | 4.55 | General pest-carrying grain |
| 42 | 12.5 | 33.6 | 27.037 | 68 | 71 | 4.23 | Serious pest-carrying grain |
| 43 | 11.8 | 34.1 | 15.278 | 20 | 19 | 5.26 | General pest-carrying grain |
| 44 | 12.1 | 33.8 | 17.793 | 29 | 26 | 11.54 | General pest-carrying grain |
| 45 | 11.6 | 34.5 | 13.957 | 16 | 16 | 0.00 | General pest-carrying grain |
| 46 | 11.8 | 34.7 | 11.397 | 10 | 9 | 11.11 | General pest-carrying grain |
| 47 | 12.1 | 34.6 | 9.637 | 7 | 6 | 16.67 | General pest-carrying grain |
| 48 | 12.0 | 34.2 | 10.745 | 9 | 8 | 12.50 | General pest-carrying grain |
| 49 | 11.7 | 33.3 | 25.076 | 61 | 57 | 7.02 | Serious pest-carrying grain |
| 50 | 12.5 | 34.8 | 8.274 | 5 | 5 | 0.00 | Basically no pest-carrying grain |
| 51 | 12.2 | 34.4 | 30.155 | 79 | 78 | 1.28 | Serious pest-carrying grain |
| 52 | 11.8 | 33.9 | 21.839 | 43 | 40 | 7.50 | Serious pest-carrying grain |
| 53 | 11.9 | 33.9 | 9.603 | 7 | 6 | 16.67 | General pest-carrying grain |
| 54 | 12.5 | 34.5 | 9.418 | 7 | 7 | 0.00 | General pest-carrying grain |
| 55 | 12.1 | 35.0 | 7.388 | 4 | 3 | 33.33 | Basically no pest-carrying grain |
| 56 | 11.7 | 33.6 | 12.978 | 15 | 14 | 7.14 | General pest-carrying grain |
| 57 | 11.9 | 33.1 | 19.230 | 37 | 34 | 8.82 | Serious pest-carrying grain |
| 58 | 11.5 | 35.0 | 9.946 | 7 | 8 | 12.50 | General pest-carrying grain |
| 59 | 12.5 | 33.9 | 7.588 | 4 | 5 | 20.00 | Basically no pest-carrying grain |
| 60 | 12.4 | 35.0 | 24.792 | 50 | 48 | 4.17 | Serious pest-carrying grain |
| 61 | 13.4 | 25.5 | 14.992 | 57 | 58 | 1.72 | Serious pest-carrying grain |
| 62 | 12.8 | 25.0 | 2.194 | 5 | 6 | 16.67 | General pest-carrying grain |
| 63 | 12.7 | 25.5 | 2.525 | 5 | 4 | 25.00 | Basically no pest-carrying grain |
| 64 | 12.6 | 25.5 | 4.533 | 12 | 13 | 7.69 | General pest-carrying grain |
| 65 | 12.7 | 26.0 | 4.070 | 8 | 9 | 11.11 | General pest-carrying grain |
| 66 | 12.6 | 25.8 | 11.088 | 37 | 40 | 7.50 | Serious pest-carrying grain |
| 67 | 13.2 | 26.0 | 3.208 | 6 | 7 | 14.29 | General pest-carrying grain |
| 68 | 13.2 | 25.3 | 2.142 | 4 | 5 | 20.00 | Basically no pest-carrying grain |
| 69 | 13.4 | 26.9 | 16.019 | 53 | 55 | 3.64 | Serious pest-carrying grain |
| 70 | 13.3 | 25.8 | 2.661 | 4 | 3 | 33.33 | Basically no pest-carrying grain |
| 71 | 13.1 | 27.0 | 13.972 | 43 | 45 | 4.44 | Serious pest-carrying grain |
| 72 | 13.1 | 25.5 | 7.145 | 22 | 24 | 8.33 | General pest-carrying grain |
| 73 | 12.8 | 26.2 | 15.875 | 57 | 63 | 9.52 | Serious pest-carrying grain |
| 74 | 12.8 | 26.6 | 7.246 | 17 | 18 | 5.56 | General pest-carrying grain |
| 75 | 13.0 | 27.3 | 8.511 | 19 | 20 | 5.00 | General pest-carrying grain |
| 76 | 13.5 | 26.4 | 2.395 | 2 | 2 | 0.00 | Basically no pest-carrying grain |
| 77 | 12.8 | 27.4 | 9.270 | 21 | 23 | 8.70 | General pest-carrying grain |
| 78 | 13.4 | 26.6 | 3.382 | 4 | 5 | 20.00 | Basically no pest-carrying grain |
| 79 | 12.9 | 25.5 | 6.893 | 21 | 22 | 4.55 | General pest-carrying grain |
| 80 | 12.9 | 25.8 | 11.632 | 39 | 41 | 4.88 | Serious pest-carrying grain |
| 81 | 12.6 | 30.0 | 6.167 | 4 | 4 | 0.00 | Basically no pest-carrying grain |
| 82 | 13.5 | 32.1 | 7.618 | 4 | 5 | 20.00 | Basically no pest-carrying grain |
| 83 | 12.7 | 30.5 | 10.160 | 14 | 13 | 7.69 | General pest-carrying grain |
| 84 | 12.6 | 32.6 | 7.659 | 4 | 4 | 0.00 | Basically no pest-carrying grain |
| 85 | 12.8 | 28.2 | 11.344 | 26 | 24 | 8.33 | General pest-carrying grain |
| 86 | 13.5 | 30.5 | 17.053 | 37 | 37 | 0.00 | Serious pest-carrying grain |
| 87 | 13.2 | 32.8 | 9.697 | 8 | 8 | 0.00 | General pest-carrying grain |
| 88 | 12.7 | 30.0 | 9.264 | 12 | 12 | 0.00 | General pest-carrying grain |
| 89 | 13.3 | 29.4 | 8.631 | 12 | 11 | 9.09 | General pest-carrying grain |
| 90 | 13.0 | 28.7 | 10.641 | 21 | 19 | 10.53 | General pest-carrying grain |
| 91 | 12.9 | 29.3 | 6.337 | 6 | 7 | 14.29 | General pest-carrying grain |
| 92 | 12.8 | 29.2 | 10.887 | 20 | 18 | 11.11 | General pest-carrying grain |
| 93 | 12.7 | 28.3 | 15.890 | 44 | 47 | 6.38 | Serious pest-carrying grain |
| 94 | 12.9 | 29.0 | 20.231 | 60 | 56 | 7.14 | Serious pest-carrying grain |
| 95 | 12.8 | 28.4 | 5.218 | 5 | 5 | 0.00 | Basically no pest-carrying grain |
| 96 | 12.7 | 28.3 | 13.047 | 32 | 33 | 3.03 | Serious pest-carrying grain |
| 97 | 13.2 | 28.6 | 17.753 | 50 | 47 | 6.38 | Serious pest-carrying grain |
| 98 | 13.4 | 32.2 | 16.365 | 27 | 28 | 3.57 | General pest-carrying grain |
| 99 | 13.3 | 32.8 | 17.396 | 28 | 31 | 9.68 | Serious pest-carrying grain |
| 100 | 13.2 | 32.8 | 15.103 | 21 | 22 | 4.55 | General pest-carrying grain |
| 101 | 12.8 | 33.5 | 9.830 | 7 | 8 | 12.50 | General pest-carrying grain |
| 102 | 12.9 | 35.0 | 18.206 | 24 | 26 | 7.69 | General pest-carrying grain |
| 103 | 12.6 | 33.8 | 20.220 | 34 | 31 | 9.68 | Serious pest-carrying grain |
| 104 | 13.0 | 34.6 | 29.313 | 65 | 60 | 8.33 | Serious pest-carrying grain |
| 105 | 13.2 | 33.5 | 24.947 | 53 | 49 | 8.16 | Serious pest-carrying grain |
| 106 | 12.8 | 35.0 | 14.366 | 14 | 15 | 6.67 | General pest-carrying grain |
| 107 | 13.4 | 33.7 | 28.953 | 69 | 66 | 4.55 | Serious pest-carrying grain |
| 108 | 13.0 | 34.5 | 15.224 | 17 | 15 | 13.33 | General pest-carrying grain |
| 109 | 12.9 | 33.8 | 16.626 | 23 | 21 | 9.52 | General pest-carrying grain |
| 110 | 13.2 | 34.7 | 22.839 | 39 | 37 | 5.41 | Serious pest-carrying grain |
| 111 | 12.8 | 34.0 | 6.758 | 2 | 3 | 33.33 | Basically no pest-carrying grain |
| 112 | 13.1 | 33.4 | 7.888 | 4 | 4 | 0.00 | Basically no pest-carrying grain |
| 113 | 13.4 | 34.5 | 12.155 | 11 | 10 | 10.00 | General pest-carrying grain |
| 114 | 13.2 | 35.0 | 5.445 | 2 | 2 | 0.00 | Basically no pest-carrying grain |
| 115 | 12.9 | 34.7 | 13.116 | 12 | 11 | 9.09 | General pest-carrying grain |
| 116 | 13.3 | 34.9 | 8.043 | 4 | 3 | 33.33 | Basically no pest-carrying grain |
| 117 | 13.5 | 34.1 | 11.753 | 10 | 9 | 11.11 | General pest-carrying grain |
| 118 | 13.3 | 35.0 | 27.214 | 54 | 51 | 5.88 | Serious pest-carrying grain |
| 119 | 12.7 | 33.9 | 17.869 | 26 | 23 | 13.04 | General pest-carrying grain |
| 120 | 12.6 | 35.0 | 10.353 | 7 | 6 | 16.67 | General pest-carrying grain |
| 121 | 13.8 | 25.0 | 4.346 | 12 | 13 | 7.69 | General pest-carrying grain |
| 122 | 14.4 | 26.2 | 5.926 | 13 | 14 | 7.14 | General pest-carrying grain |
| 123 | 14.1 | 26.8 | 3.431 | 3 | 2 | 50.00 | Basically no pest-carrying grain |
| 124 | 14.0 | 25.0 | 17.017 | 68 | 66 | 3.03 | Serious pest-carrying grain |
| 125 | 14.3 | 25.8 | 7.442 | 20 | 22 | 9.09 | General pest-carrying grain |
| 126 | 14.3 | 25.9 | 6.050 | 15 | 16 | 6.25 | General pest-carrying grain |
| 127 | 14.5 | 25.9 | 15.259 | 53 | 58 | 8.62 | Serious pest-carrying grain |
| 128 | 13.9 | 26.5 | 5.475 | 10 | 11 | 9.09 | General pest-carrying grain |
| 129 | 14.4 | 27.4 | 3.483 | 2 | 2 | 0.00 | Basically no pest-carrying grain |
| 130 | 13.7 | 25.5 | 4.930 | 12 | 14 | 14.29 | General pest-carrying grain |
| 131 | 14.0 | 25.0 | 13.862 | 52 | 49 | 6.12 | Serious pest-carrying grain |
| 132 | 13.6 | 26.2 | 6.176 | 14 | 16 | 12.50 | General pest-carrying grain |
| 133 | 13.8 | 26.0 | 14.096 | 47 | 49 | 4.08 | Serious pest-carrying grain |
| 134 | 14.2 | 25.5 | 9.188 | 28 | 29 | 3.45 | General pest-carrying grain |
| 135 | 13.9 | 27.8 | 12.042 | 28 | 31 | 9.68 | Serious pest-carrying grain |
| 136 | 14.3 | 25.2 | 2.030 | 4 | 5 | 20.00 | Basically no pest-carrying grain |
| 137 | 14.3 | 25.4 | 8.618 | 26 | 28 | 7.14 | General pest-carrying grain |
| 138 | 13.6 | 25.0 | 3.631 | 10 | 11 | 9.09 | General pest-carrying grain |
| 139 | 13.6 | 25.5 | 10.934 | 36 | 35 | 2.86 | Serious pest-carrying grain |
| 140 | 14.1 | 25.8 | 2.433 | 3 | 4 | 25.00 | Basically no pest-carrying grain |
| 141 | 13.9 | 29.2 | 8.414 | 11 | 10 | 10.00 | General pest-carrying grain |
| 142 | 14.4 | 29.5 | 15.973 | 35 | 34 | 2.94 | Serious pest-carrying grain |
| 143 | 14.0 | 32.4 | 23.969 | 52 | 55 | 5.45 | Serious pest-carrying grain |
| 144 | 14.4 | 29.0 | 14.914 | 33 | 30 | 10.00 | General pest-carrying grain |
| 145 | 14.4 | 31.7 | 14.465 | 21 | 23 | 8.70 | General pest-carrying grain |
| 146 | 14.2 | 31.2 | 22.090 | 51 | 54 | 5.56 | Serious pest-carrying grain |
| 147 | 14.3 | 30.4 | 10.066 | 12 | 11 | 9.09 | General pest-carrying grain |
| 148 | 14.5 | 31.9 | 11.523 | 12 | 14 | 14.29 | General pest-carrying grain |
| 149 | 14.2 | 28.6 | 13.183 | 29 | 28 | 3.57 | General pest-carrying grain |
| 150 | 13.8 | 29.7 | 8.550 | 10 | 10 | 0.00 | General pest-carrying grain |
| 151 | 14.3 | 29.1 | 13.094 | 26 | 25 | 4.00 | General pest-carrying grain |
| 152 | 13.8 | 30.2 | 15.469 | 30 | 28 | 7.14 | General pest-carrying grain |
| 153 | 13.7 | 32.3 | 21.503 | 43 | 46 | 6.52 | Serious pest-carrying grain |
| 154 | 14.1 | 28.7 | 12.366 | 25 | 24 | 4.17 | General pest-carrying grain |
| 155 | 14.0 | 32.3 | 17.570 | 29 | 31 | 6.45 | Serious pest-carrying grain |
| 156 | 14.0 | 31.1 | 6.514 | 2 | 4 | 50.00 | Basically no pest-carrying grain |
| 157 | 14.5 | 29.1 | 6.199 | 5 | 4 | 25.00 | Basically no pest-carrying grain |
| 158 | 13.8 | 31.7 | 12.020 | 14 | 16 | 12.50 | General pest-carrying grain |
| 159 | 13.6 | 29.7 | 7.613 | 7 | 6 | 16.67 | General pest-carrying grain |
| 160 | 14.5 | 30.0 | 5.613 | 2 | 3 | 33.33 | Basically no pest-carrying grain |
| 161 | 13.7 | 33.9 | 23.475 | 42 | 36 | 16.67 | Serious pest-carrying grain |
| 162 | 13.9 | 35.0 | 9.727 | 5 | 5 | 0.00 | Basically no pest-carrying grain |
| 163 | 14.2 | 33.2 | 15.146 | 19 | 20 | 5.00 | General pest-carrying grain |
| 164 | 14.4 | 34.2 | 19.302 | 27 | 26 | 3.85 | General pest-carrying grain |
| 165 | 13.8 | 34.0 | 17.779 | 23 | 22 | 4.55 | General pest-carrying grain |
| 166 | 14.4 | 34.1 | 27.943 | 59 | 55 | 7.27 | Serious pest-carrying grain |
| 167 | 13.6 | 33.6 | 14.355 | 16 | 15 | 6.67 | General pest-carrying grain |
| 168 | 14.4 | 34.5 | 24.345 | 43 | 38 | 13.16 | Serious pest-carrying grain |
| 169 | 14.4 | 33.0 | 26.895 | 61 | 59 | 3.39 | Serious pest-carrying grain |
| 170 | 13.8 | 33.5 | 20.144 | 32 | 35 | 8.57 | Serious pest-carrying grain |
| 171 | 13.7 | 34.0 | 22.389 | 38 | 33 | 15.15 | Serious pest-carrying grain |
| 172 | 13.9 | 33.6 | 7.611 | 3 | 2 | 50.00 | Basically no pest-carrying grain |
| 173 | 14.4 | 33.1 | 13.253 | 14 | 15 | 6.67 | General pest-carrying grain |
| 174 | 14.2 | 33.2 | 16.826 | 23 | 21 | 9.52 | General pest-carrying grain |
| 175 | 13.9 | 35.0 | 11.302 | 8 | 7 | 14.29 | General pest-carrying grain |
| 176 | 13.8 | 33.6 | 8.235 | 4 | 3 | 33.33 | Basically no pest-carrying grain |
| 177 | 13.9 | 33.2 | 18.534 | 28 | 30 | 6.67 | General pest-carrying grain |
| 178 | 14.2 | 35.0 | 8.785 | 4 | 5 | 20.00 | Basically no pest-carrying grain |
| 179 | 14.0 | 33.0 | 8.278 | 4 | 4 | 0.00 | Basically no pest-carrying grain |
| 180 | 14.2 | 35.0 | 22.990 | 36 | 35 | 2.86 | Serious pest-carrying grain |

The experimental results showed that the detection model of the population density of the Cryptolestes ferrugineus of the present disclosure had a desirable consistency between the calculation results and the detection results of the manual inspection, indicating that the detection model of the population density of the Cryptolestes ferrugineus had a high accuracy.

### Example 3

Use of a Prediction Model of a Population Density of the Stored Grain Pest Cryptolestes ferrugineus

In order to evaluate a use effect of the method for detecting a population density of Cryptolestes ferrugineus of the present disclosure in the real grain granary, a small and medium-sized simulated grain granary (CN201610044210.X) was used to conduct the simulated real granary verification. A design capacity of the simulated granary was 250 Kg of grains. In the simulated experimental verification, 200 Kg of wheat was filled. The specific implementation method included: 200 Kg of wheat of different pest-carrying grain grades (the only pest infected with stored grains was Cryptolestes ferrugineus) was placed in the simulated granary and stored under different stored grain temperatures for one week. A three-in-one and data storage-type CO₂ detector was placed, and the experimental device was airtight to monitor the respiration rate of the Cryptolestes ferrugineus, and the population density of the Cryptolestes ferrugineus was calculated. Meanwhile, according to an inspection method of stored grain pests in “GB/T 29890-2013, Technical Criterion for Grain and Oil-Seeds Storage”, the screening method, the population density of the Cryptolestes ferrugineus was manually inspected. The experimental results were shown in Table 4.

TABLE 4
Field verification of prediction results of population density
of Cryptolestes ferrugineus in grain storage environment

| | | | CO₂ | Population density (insects/kg) | | | |
|---|---|---|---|---|---|---|---|
| | Wheat | | release | | Manual | | |
| | water | Temperature | rate | Model | inspection | | |
| SN | content (%) | (° C.) | (ppm/h) | results | results | Error % | Grade |
| 1 | 11.5 | 25.0 | 9.072 | 33 | 32 | 3.13 | Serious pest-carrying grain |
| 2 | 12.5 | 26.4 | 8.446 | 24 | 26 | 7.69 | General pest-carrying grain |
| 3 | 12.2 | 27.1 | 12.816 | 39 | 41 | 4.88 | Serious pest-carrying grain |
| 4 | 11.8 | 26.9 | 3.825 | 6 | 6 | 0.00 | General pest-carrying grain |
| 5 | 12.4 | 25.7 | 2.308 | 4 | 5 | 20.00 | Basically no pest-carrying grain |
| 6 | 12.5 | 26.7 | 3.088 | 4 | 3 | 33.33 | Basically no pest-carrying grain |
| 7 | 11.8 | 27.4 | 6.046 | 11 | 13 | 15.38 | General pest-carrying grain |
| 8 | 12.2 | 26.5 | 5.246 | 11 | 12 | 8.33 | General pest-carrying grain |
| 9 | 11.7 | 28.6 | 7.625 | 13 | 11 | 18.18 | General pest-carrying grain |
| 10 | 11.8 | 32.1 | 15.676 | 28 | 32 | 12.50 | Serious pest-carrying grain |
| 11 | 12.2 | 30.0 | 11.621 | 21 | 21 | 0.00 | General pest-carrying grain |
| 12 | 11.6 | 32.7 | 10.069 | 10 | 15 | 33.33 | General pest-carrying grain |
| 13 | 11.9 | 28.5 | 6.051 | 8 | 7 | 14.29 | General pest-carrying grain |
| 14 | 11.5 | 29.4 | 7.884 | 11 | 9 | 22.22 | General pest-carrying grain |
| 15 | 11.5 | 28.6 | 22.431 | 79 | 77 | 2.60 | Serious pest-carrying grain |
| 16 | 12.1 | 32.6 | 5.287 | 1 | 2 | 50.00 | Basically no pest-carrying grain |
| 17 | 11.7 | 34.9 | 28.405 | 67 | 64 | 4.69 | Serious pest-carrying grain |
| 18 | 12.0 | 33.3 | 11.770 | 13 | 11 | 18.18 | General pest-carrying grain |
| 19 | 11.9 | 34.4 | 8.503 | 5 | 4 | 25.00 | Basically no pest-carrying grain |
| 20 | 11.6 | 33.3 | 20.435 | 41 | 39 | 5.13 | Serious pest-carrying grain |
| 21 | 12.4 | 33.4 | 24.072 | 55 | 57 | 3.51 | Serious pest-carrying grain |
| 22 | 11.9 | 34.0 | 10.316 | 9 | 7 | 28.57 | General pest-carrying grain |
| 23 | 12.4 | 34.4 | 16.464 | 23 | 22 | 4.55 | General pest-carrying grain |
| 24 | 11.9 | 33.5 | 6.647 | 3 | 2 | 50.00 | Basically no pest-carrying grain |
| 25 | 12.9 | 26.6 | 9.432 | 26 | 27 | 3.70 | General pest-carrying grain |
| 26 | 13.0 | 25.4 | 8.527 | 28 | 28 | 0.00 | General pest-carrying grain |
| 27 | 13.1 | 25.0 | 5.559 | 18 | 16 | 12.50 | General pest-carrying grain |
| 28 | 12.7 | 25.8 | 3.475 | 7 | 9 | 22.22 | General pest-carrying grain |
| 29 | 13.0 | 25.0 | 13.537 | 53 | 51 | 3.92 | Serious pest-carrying grain |
| 30 | 12.6 | 25.4 | 8.074 | 26 | 26 | 0.00 | General pest-carrying grain |
| 31 | 13.3 | 26.2 | 1.955 | 1 | 2 | 50.00 | Basically no pest-carrying grain |
| 32 | 12.6 | 25.0 | 1.736 | 4 | 3 | 33.33 | Basically no pest-carrying grain |
| 33 | 13.1 | 29.0 | 13.317 | 30 | 28 | 7.14 | General pest-carrying grain |
| 34 | 13.2 | 28.9 | 18.949 | 54 | 51 | 5.88 | Serious pest-carrying grain |
| 35 | 13.2 | 28.0 | 6.267 | 9 | 4 | 125.00 | Basically no pest-carrying grain |
| 36 | 12.7 | 31.6 | 11.896 | 16 | 18 | 11.11 | General pest-carrying grain |
| 37 | 13.1 | 31.6 | 7.784 | 5 | 4 | 25.00 | Basically no pest-carrying grain |
| 38 | 13.2 | 32.5 | 10.941 | 11 | 13 | 15.38 | General pest-carrying grain |
| 39 | 13.3 | 28.2 | 5.024 | 5 | 5 | 0.00 | Basically no pest-carrying grain |
| 40 | 13.1 | 28.8 | 19.704 | 58 | 56 | 3.57 | Serious pest-carrying grain |
| 41 | 12.8 | 34.0 | 25.751 | 53 | 55 | 3.64 | Serious pest-carrying grain |
| 42 | 12.9 | 34.7 | 27.085 | 55 | 57 | 3.51 | Serious pest-carrying grain |
| 43 | 13.0 | 35.0 | 14.342 | 14 | 12 | 16.67 | General pest-carrying grain |
| 44 | 12.6 | 34.7 | 8.756 | 5 | 5 | 0.00 | Basically no pest-carrying grain |
| 45 | 12.7 | 33.7 | 11.179 | 10 | 9 | 11.11 | General pest-carrying grain |
| 46 | 13.1 | 33.0 | 15.672 | 22 | 19 | 15.79 | General pest-carrying grain |
| 47 | 13.4 | 33.9 | 5.917 | 1 | 0 | 0.00 | Basically no pest-carrying grain |
| 48 | 13.4 | 33.8 | 21.952 | 40 | 37 | 8.11 | Serious pest-carrying grain |
| 49 | 14.3 | 27.6 | 13.058 | 33 | 36 | 8.33 | Serious pest-carrying grain |
| 50 | 13.7 | 25.7 | 14.513 | 51 | 53 | 3.77 | Serious pest-carrying grain |
| 51 | 14.3 | 25.0 | 3.862 | 10 | 10 | 0.00 | General pest-carrying grain |
| 52 | 14.5 | 25.1 | 2.220 | 5 | 4 | 25.00 | Basically no pest-carrying grain |
| 53 | 13.7 | 27.6 | 4.264 | 4 | 3 | 33.33 | Basically no pest-carrying grain |
| 54 | 14.0 | 25.9 | 9.461 | 27 | 29 | 6.90 | General pest-carrying grain |
| 55 | 14.3 | 27.7 | 8.865 | 17 | 16 | 6.25 | General pest-carrying grain |
| 56 | 14.1 | 26.5 | 7.626 | 18 | 19 | 5.26 | General pest-carrying grain |
| 57 | 13.6 | 32.6 | 9.970 | 8 | 9 | 11.11 | General pest-carrying grain |
| 58 | 14.5 | 28.5 | 23.509 | 76 | 77 | 1.30 | Serious pest-carrying grain |
| 59 | 14.2 | 30.0 | 7.840 | 7 | 6 | 16.67 | General pest-carrying grain |
| 60 | 13.9 | 31.5 | 14.600 | 22 | 25 | 12.00 | General pest-carrying grain |
| 61 | 13.6 | 32.3 | 17.608 | 29 | 30 | 3.33 | General pest-carrying grain |
| 62 | 14.3 | 32.1 | 18.802 | 34 | 36 | 5.56 | Serious pest-carrying grain |
| 63 | 14.3 | 31.8 | 11.247 | 12 | 14 | 14.29 | General pest-carrying grain |
| 64 | 14.1 | 32.2 | 6.099 | 1 | 2 | 50.00 | Basically no pest-carrying grain |
| 65 | 13.8 | 33.9 | 25.157 | 49 | 47 | 4.26 | Serious pest-carrying grain |
| 66 | 13.8 | 33.5 | 10.901 | 8 | 7 | 14.29 | General pest-carrying grain |
| 67 | 13.9 | 35.0 | 22.300 | 34 | 35 | 2.86 | Serious pest-carrying grain |
| 68 | 13.7 | 33.2 | 16.820 | 23 | 22 | 4.55 | General pest-carrying grain |
| 69 | 14.4 | 33.5 | 28.609 | 66 | 63 | 4.76 | Serious pest-carrying grain |
| 70 | 14.3 | 35.0 | 7.302 | 3 | 3 | 0.00 | Basically no pest-carrying grain |
| 71 | 13.7 | 33.2 | 14.405 | 17 | 15 | 13.33 | General pest-carrying grain |
| 72 | 14.1 | 35.0 | 9.185 | 5 | 5 | 0.00 | Basically no pest-carrying grain |

The results of practical application in the simulated granary showed that the prediction results of the population density of the Cryptolestes ferrugineus had an extremely desirable consistency with the result of manual inspection, indicating that the method for detecting a population density of Cryptolestes ferrugineus of the present disclosure was accurate, simple and efficient.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A method for detecting a population density of Cryptolestes ferrugineus based on a CO₂ release rate, comprising:
constructing a prediction model of a population density of Cryptolestes ferrugineus based on a relationship between different stored grain temperatures, stored grain water contents, as well as CO₂ release rates in the environment and the population density of the Cryptolestes ferrugineus; and
measuring the stored grain temperature and the CO₂ release rate in the environment, substituting measured values into the prediction model of the population density of the Cryptolestes ferrugineus for calculation to obtain the population density of the Cryptolestes ferrugineus in a grain storage environment.

2-10. (canceled)

11. The method for detecting a population density of Cryptolestes ferrugineus based on a CO₂ release rate according to claim 1, wherein when the stored grain water content is 11.5% to 12.5%, the prediction model of the population density of the Cryptolestes ferrugineus is shown in formula I: 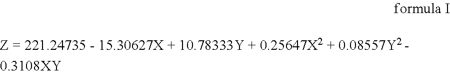
wherein, R²=0.94022; X is the temperature in ° C.; Y is the CO₂ release rate in ppm/h; and Z is the population density of the Cryptolestes ferrugineus in insects/kg.

12. The method for detecting a population density of Cryptolestes ferrugineus based on a CO₂ release rate according to claim 1, wherein when the stored grain water content is 12.6% to 13.5%, the prediction model of the population density of the Cryptolestes ferrugineus is shown in formula II: 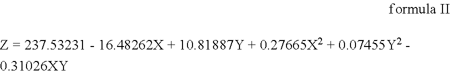
wherein, R²=0.93665; X is the temperature in ° C.; Y is the CO₂ release rate in ppm/h; and Z is the population density of the Cryptolestes ferrugineus in insects/kg.

13. The method for detecting a population density of Cryptolestes ferrugineus based on a CO₂ release rate according to claim 1, wherein when the stored grain water content is 13.6% to 14.5%, the prediction model of the population density of the Cryptolestes ferrugineus is shown in formula III: 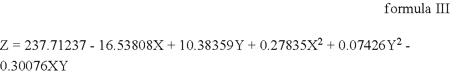
wherein, R²=0.93604; X is the temperature in ° C.; Y is the CO₂ release rate in ppm/h; and Z is the population density of the Cryptolestes ferrugineus in insects/kg.

14. The method for detecting a population density of Cryptolestes ferrugineus based on a CO₂ release rate according to claim 1, wherein the temperature comprises 25° C. to 35° C.

15. The method for detecting a population density of Cryptolestes ferrugineus based on a CO₂ release rate according to claim 11, wherein the temperature comprises 25° C. to 35° C.

16. The method for detecting a population density of Cryptolestes ferrugineus based on a CO₂ release rate according to claim 12, wherein the temperature comprises 25° C. to 35° C.

17. The method for detecting a population density of Cryptolestes ferrugineus based on a CO₂ release rate according to claim 13, wherein the temperature comprises 25° C. to 35° C.

18. The method for detecting a population density of Cryptolestes ferrugineus based on a CO₂ release rate according to claim 14, wherein in the Cryptolestes ferrugineus, adults and larvae are at a quantity ratio of 1:(4.93-8.37).

19. The method for detecting a population density of Cryptolestes ferrugineus based on a CO₂ release rate according to claim 15, wherein in the Cryptolestes ferrugineus, adults and larvae are at a quantity ratio of 1:(4.93-8.37).

20. The method for detecting a population density of Cryptolestes ferrugineus based on a CO₂ release rate according to claim 16, wherein in the Cryptolestes ferrugineus, adults and larvae are at a quantity ratio of 1:(4.93-8.37).

21. The method for detecting a population density of Cryptolestes ferrugineus based on a CO₂ release rate according to claim 17, wherein in the Cryptolestes ferrugineus, adults and larvae are at a quantity ratio of 1:(4.93-8.37).

22. The method for detecting a population density of Cryptolestes ferrugineus based on a CO₂ release rate according to claim 1, wherein the stored grain comprises wheat.

23. The method for detecting a population density of Cryptolestes ferrugineus based on a CO₂ release rate according to claim 22, wherein a pest in the stored grain is the Cryptolestes ferrugineus.

24. A method for determining a pest-carrying grain grade by using a method for detecting a population density of Cryptolestes ferrugineus based on a CO₂ release rate.

25. The method according to claim 24, wherein the pest-carrying grain grade comprises a grade of basically no pest-carrying grain, a grade of general pest-carrying grain, and a grade of serious pest-carrying grain in unprocessed wheat grains.
